(19) United States
(12) Patent Application Publication
(10) Pub. No.: US 2006/0134806 A1
(43) Pub. Date: Jun. 22, 2006

(54) Method of separating unattached Raman-active tag from bioassay or other reaction mixture

(75) Inventor: Frank John Mondello, Niskayuna, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 11/018,512

(22) Filed: Dec. 20, 2004

(51) Int. Cl. G01N 33/551

(52) U.S. Cl. 436/524; 977/904

(57) ABSTRACT

A method of separating a Raman-active tag unattached to a target from a Raman-active complex is disclosed. The Raman-active complex includes a Raman-active tag attached to a target. The method includes providing a mixture including at least one Raman-active tag unattached to a target and at least one Raman-active complex; and passing the mixture through a porous membrane.

### BACKGROUND OF THE INVENTION

The invention relates to Raman-active bioassays or other reaction mixture. Particularly, the invention is directed to a method of separating an unattached Raman-active tag from a bioassay or other reaction mixture.

### DESCRIPTION OF RELATED ART

Raman-active tags 100 are known to detect the presence of pathogenic organisms or other materials. FIG. 1 is a schematic representation of a Raman-active tag 100 that includes a Raman-active particle 110 and one or more target-binding moieties 112. The target-binding moiety 112 on the Raman-active tag 100 is configured to allow the Raman-active tag 100 to attach to one or more targets 212 to form a Raman-active complex 200. In contrast, the Raman-active tag 100 is unattached to a target 212. FIG. 2 and FIG. 3 are schematic representations of a Raman-active complex 200 comprising a Raman-active tag 100 and a target 212. In the presence of a target 212, one or more target-binding moieties 112 against a target 212 allow the Raman-active tag 100 to attach to the target 212. Detection of the target 212 is then based on the presence of a Raman signal after removing any Raman-active tags 100 that are unattached to a target 212 from the test mixture. Failure to eliminate unattached Raman-active tags 100 results in false positive detection of the presence of the target 212. Centrifugation is a method commonly used to separate unattached Raman-active tags 100 from Raman active complexes 200 that are attached to a target; however, centrifugation is inefficient because the Raman-active tags 100 have a density such that the Raman-active tags 100 pellet along with the Raman active complexes 200 and targets 212.

Thus, a need still remains for a method of separating unattached Raman-active tag 100 from bioassay or other reaction mixture.

### SUMMARY

The purpose and advantages of embodiments of the invention will be set forth and apparent from the description that follows, as well as will be learned by practice of the embodiments of the invention. Additional advantages will be realized and attained by the methods and systems particularly pointed out in the written description and claims hereof, as well as from the appended drawings. An embodiment of the invention provides a method of separating a Raman-active tag unattached to a target from a Raman-active complex. The Raman-active complex comprises a Raman-active tag attached to a target. The method comprises providing a mixture comprising at least one Raman-active tag unattached to a target and at least one Raman-active complex; and passing the mixture through a porous membrane. Each Raman-active complex has a first size and the membrane has pores with an effective size smaller than the first size.

A second embodiment provides a method of separating a Raman-active tag unattached to a target from a Raman-active complex. The Raman-active complex comprises a Raman-active tag attached to a target. The method comprises providing a mixture comprising at least one Raman-active tag unattached to a target and at least one Raman-active complex; passing the mixture through a porous membrane; and applying a sufficient force to the porous membrane to separate at least one Raman-active complex from the membrane while the Raman-active tag unattached to a target remains on the membrane. Each Raman-active complex has a first size and the membrane has pores with an effective size smaller than the first size.

A third embodiment provides a method of separating a Raman-active tag unattached to a target from a Raman-active complex. The Raman-active complex comprises a Raman-active tag attached to a target. The method comprises providing a mixture comprising at least one Raman-active tag unattached to a target and at least one Raman-active complex; and passing the mixture through a porous membrane comprising pores, wherein the porous membrane is configured to collect at least one Raman-active complex. Each Raman-active complex has a first size.

The accompanying figures, which are incorporated in and constitute part of this specification, are included to illustrate and provide a further understanding of the method and system of the invention. Together with the description, the drawings serve to explain the principles of the invention.

### BRIEF DESCRIPTION OF THE DRAWINGS

FIG. 1 is a schematic representation of an unattached Raman-active tag in accordance with an embodiment of the invention;

FIG. 2 is a schematic representation of a Raman-active complex in accordance with an embodiment of the invention;

FIG. 3 is a schematic representation of Raman-active complex in accordance with an embodiment of the invention;

FIG. 4 is a schematic representation of a method of separating a Raman-active tag unattached to a target from a Raman-active complex in accordance with an embodiment of the invention;

FIG. 5 is a flow chart of a method of separating a Raman-active tag unattached to a target from a Raman-active complex in accordance with an embodiment of the invention;

FIG. 6A is a Raman spectrum of a sample of Bacillus spores not exposed to Raman-active tags in accordance with an embodiment of the invention;

FIG. 6B is a Raman spectrum of a sample of Raman-active tags functionalized with anti-bacillus antibodies filtered in the absence of bacillus spores in accordance with an embodiment of the invention;

FIG. 6C is a Raman spectrum of a sample of Bacillus spores exposed to Raman-active tags in accordance with an embodiment of the invention;

FIG. 6D is a Raman spectrum of a sample of Bacillus spores exposed to Raman-active tags designed to detect E. coli cells in accordance with an embodiment of the invention;

FIG. 6E is a Raman spectrum of control samples of the antibody functionalized Raman tags placed directly on a glass surface in accordance with an embodiment of the invention;

FIG. 7A is a Raman spectrum of a sample of E. coli cells not exposed to Raman-active tags in accordance with an embodiment of the invention

FIG. 7B is a Raman spectrum of a sample of Raman-active tags functionalized with anti-E. coli antibodies filtered in the absence of E. coli cells in accordance with an embodiment of the invention;

FIG. 7C is a Raman spectrum of a sample of E. coli cells exposed to Raman-active tags bound to anti-E. coli antibody in accordance with an embodiment of the invention;

FIG. 7D is a Raman spectrum of a sample of E. coli cells exposed to Raman-active tags designed to detect Bacillus spores in accordance with an embodiment of the invention; and

FIG. 7E is a Raman spectrum of control samples of the antibody functionalized Raman tags placed directly on a glass surface in accordance with an embodiment of the invention.

### DETAILED DESCRIPTION OF EXEMPLARY EMBODIMENTS

Reference will now be made in detail to exemplary embodiments of the invention, which are illustrated in the accompanying figures and examples. Referring to the drawings in general, it will be understood that the illustrations are for the purpose of describing a particular embodiment of the invention and are not intended to limit the invention thereto.

Whenever a particular embodiment of the invention is said to comprise or consist of at least one element of a group and combinations thereof, it is understood that the embodiment may comprise or consist of any of the elements of the group, either individually or in combination with any of the other elements of that group. Furthermore, when any variable occurs more than one time in any constituent or in formula, its definition on each occurrence is independent of its definition at every other occurrence. Also, combinations of substituents and/or variables are permissible only if such combinations result in stable compounds.

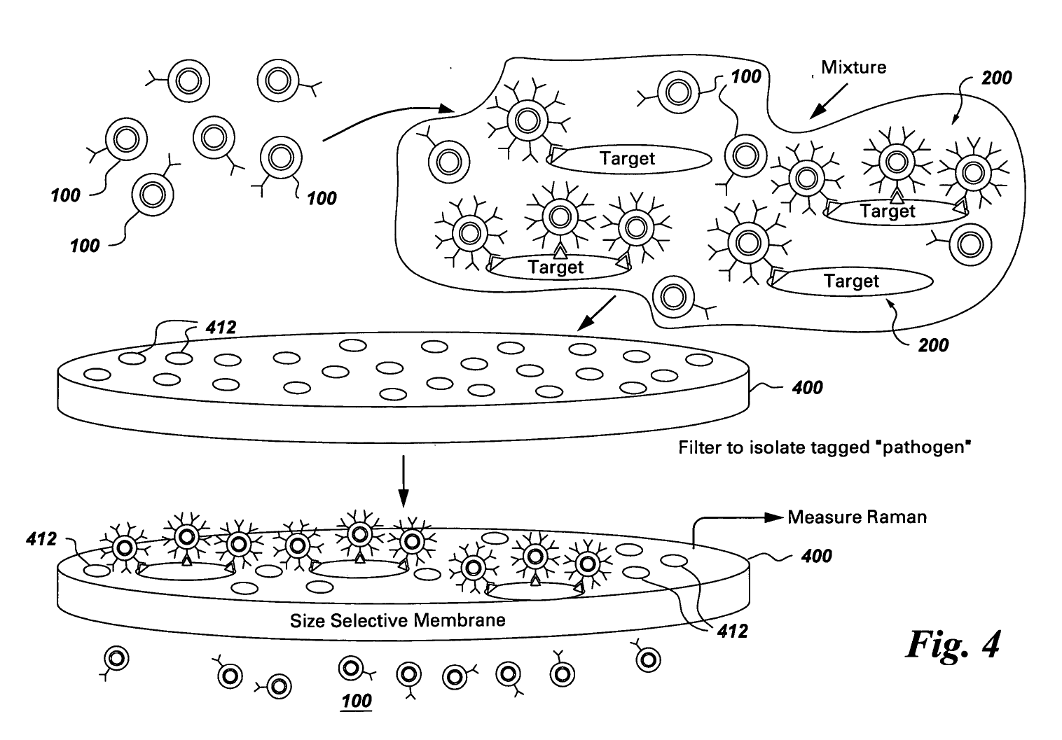

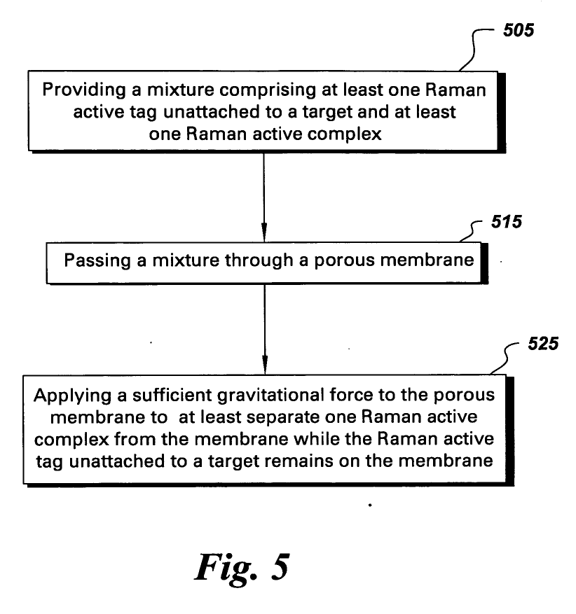

With reference to FIG. 4 and FIG. 5, an embodiment of a method of separating one or more Raman-active tags 100 which are unattached to a target 212 from one or more Raman-active complexes 200 is described. FIG. 4 is a schematic representation of a method of separating one or more Raman-active tags 100 unattached to a target 212 from one or more Raman-active complexes 200. FIG. 5 is a flow chart of a method of separating one or more Raman-active tags 100 unattached to a target 212 from one or more Raman-active complexes 200.

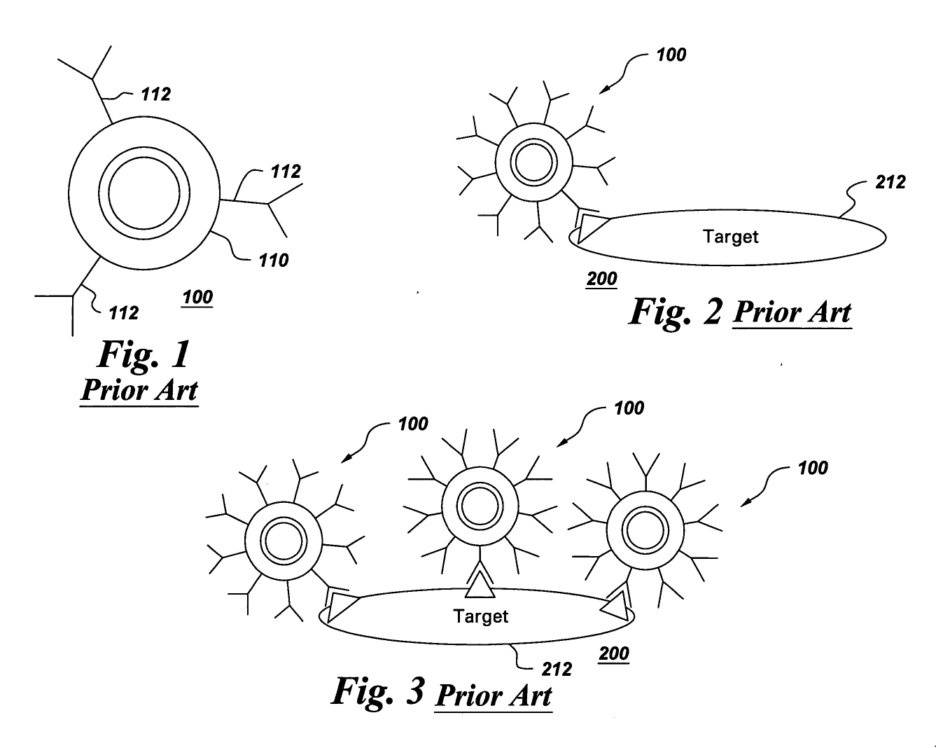

The method includes, at Step 505, providing a mixture comprising at least one Raman-active tag 100 and at least one Raman-active complex 200. In one embodiment, the Raman-active tag 100 is immuno-functionalized. Immuno-functionalized Raman-active tags 100 detect the presence of one or more targets 212 that are pathogenic organisms or other materials. Immuno-functionalized Raman-active tags 100 include Raman-active tags 100 attached to one or more target-binding moieties 112 that are antibodies. The target-binding moiety 112 is configured to allow the Raman-active tag 100 to attach to a target 212 to form a Raman-active complex 200. Attached means the target-binding moiety 112 is covalently or non-covalently connected to a target 212. Examples of target-binding moieties 112 include, but are not limited to, antibodies, aptamers, polypeptides, peptide nucleic acids, avidin, streptavidin, and derivatives of avidin and streptavidin. The Raman-active tag 100 may comprise one target-binding moiety 112 or a plurality of target-binding moieties 112, as in FIG. 1. The plurality of target-binding moieties 112 may all be of the same kind of target-binding moieties 112 or different kinds of target-binding moieties 112.

Examples of targets 212 to which a target-binding moiety 112 may attach include, but are not limited to, organisms such as viruses, bacteria, yeast, spores, liposomes, and beads. Examples of beads include, but are not limited to, latex, polystyrene, silica and plastic. In one embodiment, a target 212 is attached to one Raman-active complex 200 as in FIG. 2 or a plurality of Raman-active complexes 200 as in FIG. 3. In another embodiment, target-binding moieties 112 include antibodies and targets 212 include bacteria.

The Raman-active complex 200 has a first size. First size means any size larger than a target 212 alone. In one embodiment, the Raman-active tag 100 unattached to a target 212 has a second size smaller than the first size. In another embodiment, the first size is at least twice the second size. In yet another embodiment, the first size is at least five times the second size. In one embodiment, the first size is in a range from about 500 nm to about 1000 nm and the second size is in a range from about 10 nm to about 100 nm.

Next, Step 515 comprises passing the mixture through a porous membrane 400. Examples of the porous membrane 400 include, but are not limited to, cellulose acetate, glass fiber, vinyl, poly vinylidene fluoride, and nitrocellulose.

In one embodiment of the Step 515 of passing the mixture through the porous membrane 400, the porous membrane 400 has pores 412 with a maximum pore size smaller than the first size. In yet another embodiment, the pores 412 of the porous membrane 400 have a maximum pore size larger than the second size. In another embodiment, the pores 412 have a maximum pore size of about two to about five times smaller than the first size. In another embodiment, the pores 412 have a maximum pore size in a range from about 0.1 μm to about 5 μm.

In another embodiment of the Step 515 of passing the mixture through the porous membrane 400, the porous membrane is configured to collect at least one Raman-active complex 200.

The method may further optionally comprise the Step 525 of applying a sufficient force to the porous membrane 400 to separate at least one Raman-active complex 200 from the porous membrane 400 while the Raman-active tag 100 unattached to a target 212 remains on the porous membrane 400. In one embodiment, applying a sufficient force to the porous membrane 400 comprising vortexing the porous membrane 400 in a buffer, or otherwise washing or rinsing the porous membrane 400 such as to remove the Raman-active complexes 200.

The method may also further comprise taking a Raman spectrum of the Raman-active complex 200 collected on the porous membrane 400. The Raman spectrum may be taken directly on the porous membrane 400 without removing the Raman-active complex 200 from the porous membrane 400 or from the mixture collected on the porous membrane 400 after being removed from the porous membrane 400.

The following examples serve to illustrate the features and advantages of the invention and are not intended to limit the invention thereto.

A sample of target microorganisms 212, which includes but is not restricted to bacteria, spores, and viruses, was added to a sample container such as an eppindorf tube. The sample was then pelleted by centrifugation and resuspended in a buffer solution. The sample was then again pelleted by centrifugation and resuspended in a buffer solution. Immuno-functionalized Raman-active tags 100 were added to the target microorganisms 212 and mixed to form a mixture. The mixture was then incubated on ice to form Raman-active complexes 200. A buffer solution was then added to the mixture and any unattached Raman-active tags 100 were separated from the Raman active complex 200 using a porous membrane 400 (also referred as filter). After filtration, the filter 400 was placed into a tube-containing buffer and placed on a vortex mixer at top speed to dislodge the Raman-active complex 200 from the filter 400. The liquid comprising the Raman active complex 200 and any unattached target microorganisms 212 was then transferred to a tube and pelleted by centrifugation. After removal of the supernatant fluid, the sample is re-suspended in a small volume of buffer and spotted onto a sample holder for Raman analysis.

Examples 1 and 2 below demonstrate that it is possible to use immuno-functionalized Raman-active tags 100 to detect the presence of a specific target organism 212. In these experiments, a Raman signal was only detected when the appropriate target organism 212 and Raman-active tags 100 immuno-functionalized for that specific target organism 212 to detect the presence of that specific target organism 212 were both present.

### EXAMPLE 1

Example 1 demonstrates that it is possible to use immuno-functionalized Raman-active tags 100 to detect the presence of Bacillus spores. The Raman-active tags 100 were specifically immuno-functionalized to detect the presence of Bacillus spores by attachment of anti-bacillus antibodies (target-binding moieties 112). A Raman signal was only detected when the Bacillus spores and Raman-active tags 100 comprising the anti-bacillus antibody (target-binding moieties 112) in combination was present.

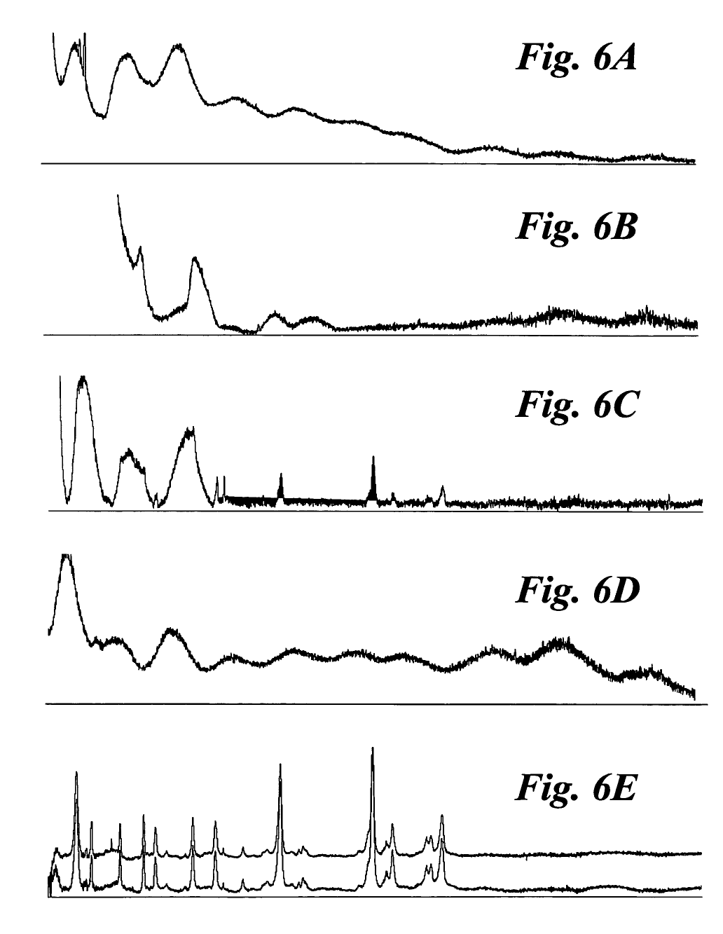

FIG. 6A is a Raman spectrum of a sample of Bacillus spores not exposed to Raman-active tags 100. As expected, in the absence of any Raman-active tags 100, there was no detectable Raman signal.

FIG. 6B is a Raman spectrum of a sample of Raman-active tags 100 functionalized with anti-bacillus antibodies (target-binding moiety 112) filtered in the absence of bacillus spores. In the absence of the target Bacillus spores 212, the Raman-active tags 100 did not form Raman-active complex 200 and were not retained by the filter 400. Consequently, there was no detectable Raman signal from the material collected on the filter 400.

FIG. 6C is a Raman spectrum of a sample of target Bacillus spores 212 exposed to Raman-active tags 100 that are immuno-functionalized with anti-bacillus antibodies (target-binding moieties 112). The Raman-active tags 100 immuno-functionalized with anti-bacillus antibodies (target-binding moiety 112) attached to the target Bacillus spores 212 and formed Raman-active complex 200, which were then retained by the filter 400. Consequently, there was a detectable Raman signal from the material collected on the filter 400.

FIG. 6D is a Raman spectrum of a sample of target Bacillus spores 212 exposed to Raman-active tags 100 immuno-functionalized with anti-E. coli antibodies, as opposed to ant-Bacillus antibodies, to detect target Bacillus spores 212. The Raman-active tags 100 immuno-functionalized with anti-E. coli antibodies instead of anti-Bacillus antibodies did not attach to the target Bacillus spores 212 to form Raman-active complex 200 and passed though the filter 400. Consequently, there was no detectable Raman signal from the material collected on the filter 400

FIG. 6E is a Raman spectrum of control samples of Raman tags 100 immuno-functionalized with anti-Bacillus antibodies placed directly on a glass surface. This is a positive control that shows the expected Raman spectrum of the Raman-active particle 110.

### EXAMPLE 2

Example 2 demonstrates that it is possible to use immuno-functionalized Raman-active tags 100 to detect the presence of E. coli cells. The Raman-active tags 100 were specifically immuno-functionalized to detect the presence of E. coli cells by attachment of anti-E. coli antibodies (target-binding moieties 112). A Raman signal was only detected when E. coli and Raman-active tags 100 comprising the anti-E. coli antibodies (target-binding moieties 112) in combination was present

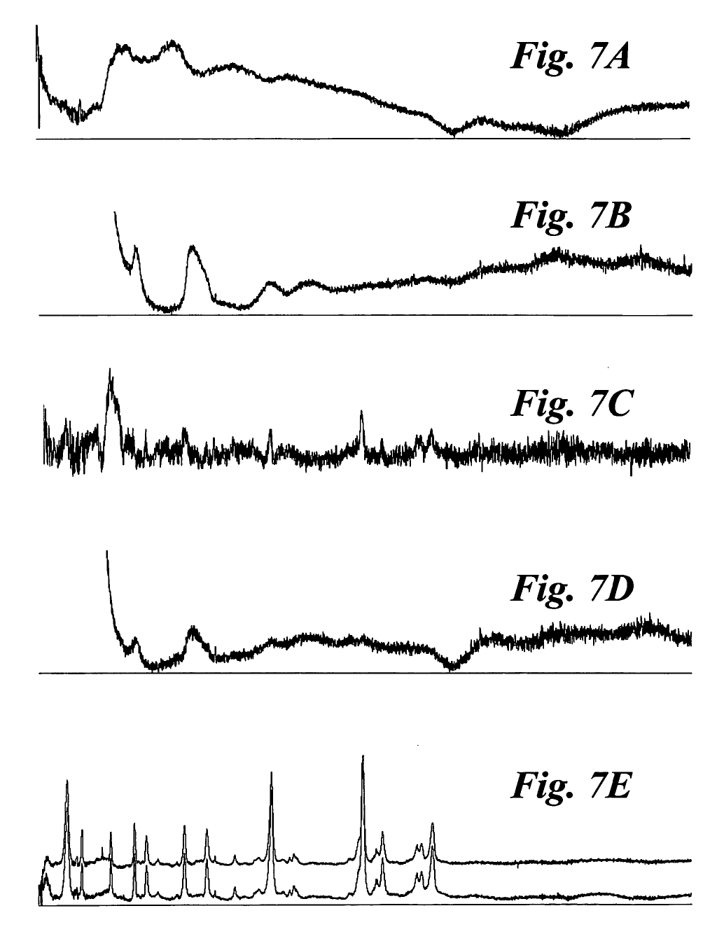

FIG. 7A is a Raman spectrum of a sample of E. coli cells not exposed to Raman-active tags 100. As expected, in the absence of any Raman-active tags 100, there was no detectable Raman signal

FIG. 7B is a Raman spectrum of a sample of Raman-active tags 100 functionalized with anti-E. coli antibodies (target-binding moieties 112) filtered in the absence of target E. coli cells 212. In the absence of the target E. coli cells 212, the Raman-active tags 100 did not form Raman-active complex 200 and were not retained by the filter 400. Consequently, there was no detectable Raman signal from the material collected on the filter 400.

FIG. 7C is a Raman spectrum of a sample of target E. coli cells 212 exposed to Raman-active tags 100 that are immuno-functionalized with anti-E. coli antibodies (target-binding moieties 112). The Raman-active tags 100 immuno-functionalized with anti-E. coli antibodies (target-binding moieties 112) attached to the target E. coli cells 212 and formed Raman-active complex 200, which were then retained by the filter 400. Consequently, there was a detectable Raman signal from the material collected on the filter 400.

FIG. 7D is a Raman spectrum of a sample of target E. coli cells 212 exposed to Raman-active tags 100 immuno-functionalized with anti-Bacillus antibodies (target-binding moieties 112), as opposed to anti-E. coli antibodies, to detect the target E. coli cells 212. The Raman-active tags 100 immuno-functionalized with anti-Bacillus antibodies (target-binding moieties 112) instead of anti-E. coli antibodies did not attach to the target E. coli cells 212 to form Raman-active complex 200 and passed though the filter 400. Consequently, there was no detectable Raman signal from the material collected on the filter 400

FIG. 7E is a Raman spectrum of control samples of Raman tags 100 immuno-functionalized with anti-E. coli antibodies placed directly on a glass surface. This is a positive control that shows the expected Raman spectrum of the Raman-active particle 110.

While the invention has been described in detail in connection with only a limited number of aspects, it should be readily understood that the invention is not limited to such disclosed aspects. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed is:

1. A method of separating a Raman-active tag unattached to a target from a Raman-active complex, wherein the Raman-active complex comprises a Raman-active tag attached to a target,
the method comprising:
i) providing a mixture comprising at least one Raman-active tag unattached to a target and at least one Raman-active complex,
wherein the Raman-active complex has a first size;
ii) passing the mixture through a porous membrane,
wherein the membrane comprises pores having an effective size smaller than the first size.

2. The method of claim 1, further comprising applying a sufficient force to the porous membrane to at least separate one Raman-active complex from the membrane while the Raman-active tag unattached to a target remains on the membrane.

3. The method of claim 2, wherein applying a sufficient force to the membrane comprising vortexing the membrane in a liquid.

4. The method of claim 1, wherein the Raman-active tag comprises a Raman-active particle and a target-binding moiety, wherein the target-binding moiety is configured to attach the Raman-active particle to a target.

5. The method of claim 4, wherein the target-binding moiety comprises at least one chemical moiety selected from a group consisting of antibodies, aptamers, and polypeptides.

6. The method of claim 1, further comprising taking a Raman spectrum of the Raman-active complex collected on the membrane.

7. The method of claim 1, wherein the target comprises at least one target selected from a group consisting of viruses, bacteria, and spores.

8. The method of claim 1, wherein the target is attached to a plurality of Raman-active complexes.

9. The method of claim 1, wherein the pores of the membrane have a maximum pore size of about two to about five times smaller than the first size.

10. The method of claim 1, wherein the pores of the membrane have a maximum pore size in a range from about 0.1 μm to about 5 μm.

11. The method of claim 1, wherein the Raman-active tag unattached to a target has a second size smaller than the first size.

12. The method of claim 11, wherein the pores of the membrane have a maximum pore size larger than the second size.

13. The method of claim 11, wherein the first size is at least twice the second size.

14. The method of claim 13, wherein the first size is at least five times the second size.

15. The method of claim 11, wherein the first size is in a range from about 500 nm to about 1000 nm and the second size is in a range from about 10 nm to about 100 nm.

16. A method of separating a Raman-active tag unattached to a target from a Raman-active complex, wherein the Raman-active complex comprises a Raman-active tag attached to a target,
the method comprising:
i) providing a mixture comprising at least one Raman-active tag unattached to a target and at least one Raman-active complex,
wherein the Raman-active complex has a first size;
ii) passing the mixture through a porous membrane,
wherein the membrane comprises pores having an effective size smaller than the first size.
iii) applying a sufficient force to the membrane to at least separate one Raman-active complex from the membrane while the Raman-active tag unattached to a target remains on the membrane.

17. The method of claim 16, wherein applying a sufficient force to the membrane comprising vortexing the membrane in a liquid.

18. The method of claim 16, wherein the Raman-active tag comprises a Raman-active particle and a target-binding moiety, wherein the target-binding moiety is configured to attach the Raman-active particle to a target.

19. The method of claim 18, wherein the target-binding moiety comprises at least one chemical moiety selected from a group consisting of antibodies, aptamers, and polypeptides.

20. The method of claim 16, further comprising taking a Raman spectrum of the Raman-active complex collected on the membrane.

21. The method of claim 16, wherein the target comprises at least one target selected from a group consisting of viruses, bacteria, and spores.

22. The method of claim 16, wherein the target is attached to a plurality of Raman-active complexes.

23. The method of claim 16, wherein the pores have a maximum pore size of about two to about five times smaller than the first size.

24. The method of claim 16, wherein the pores have a maximum pore size in a range from about 0.1 μm to about 5 μm.

25. The method of claim 16, wherein the Raman-active tag unattached to a target has a second size smaller than the first size.

26. The method of claim 25, wherein the pores of the membrane have a maximum pore size larger than the second size.

27. The method of claim 25, wherein the first size is at least twice the second size.

28. The method of claim 27, wherein the first size is at least five times the second size.

29. The method of claim 25, wherein the first size is in a range from about 500 nm to about 1000 nm and the second size is in a range from about 10 nm to about 100 nm.

30. A method of separating a Raman-active tag unattached to a target from a Raman-active complex, wherein the Raman-active complex comprises a Raman-active tag attached to a target,
the method comprising:
i) providing a mixture comprising at least one Raman-active tag unattached to a target and at least one Raman-active complex,
wherein the Raman-active complex has a first size;
ii) passing the mixture through a porous membrane comprising pores,
wherein the porous membrane is configured to collect at least one Raman-active complex.

31. The method of claim 30, further comprising applying a sufficient force to the porous membrane to at least separate one Raman-active complex collected on the membrane while the Raman-active tag unattached to a target remains collected on the membrane.

32. The method of claim 31 wherein applying a sufficient force to the membrane comprising vortexing the membrane.

33. The method of claim 30, wherein the Raman-active tag comprises a Raman-active particle and a target-binding moiety, wherein the target-binding moiety is configured to attach the Raman-active particle to a target.

34. The method of claim 33, wherein the target-binding moiety comprises at least one chemical moiety selected from a group consisting of antibodies, aptamers, and polypeptides.

35. The method of claim 30, further comprising taking a Raman spectrum of the Raman-active complex collected on the membrane.

36. The method of claim 30, wherein the target comprises at least one target selected from a group consisting of viruses, bacteria and spores.

37. The method of claim 30, wherein the target is attached to a plurality of Raman-active complexes.

38. The method of claim 30, wherein the pores have a maximum pore size of about two to about five times the first size.

39. The method of claim 30, wherein the pores have an average size in a range from about 0.1 μm to about 5 μm.

40. The method of claim 30, wherein the Raman-active tag unattached to a target has a second size smaller than the first size.

41. The method of claim 40, wherein the pores of the membrane have an effective size larger than the second size.

42. The method of claim 40, wherein the first size is at least twice the second size.

43. The method of claim 42, wherein the first size is at least five times the second size.

44. The method of claim 40, wherein the first size is in a range from about 500 nm to about 1000 nm and the second size is in a range from about 10 nm to about 100 nm.
